(19) United States
(12) Patent Application Publication
(10) Pub. No.: US 2005/0283846 A1
(43) Pub. Date: Dec. 22, 2005

(54) Method for evaluating therapeutic agent for dermatitis and/or alopecia, therapeutic agent for dermatitis and/or alopecia, and transgenic mouse

(75) Inventors: Tomoaki Hoshino, Chikushino-shi (JP); Yusuke Kawase, Ashiya-shi (JP); Keiko Nomiyama, Kitakyusyu-shi (JP); Koichi Yokota, Osaka (JP); Kohichiro Yoshino, Osaka (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: Hoshino TOMOAKI

(21) Appl. No.: 11/210,692

(22) Filed: Aug. 25, 2005

Related U.S. Application Data
(63) Continuation of application No. 10/695,875, filed on Oct. 30, 2003.

(30) Foreign Application Priority Data
Jan. 30, 2003 (JP) 2003-22876
Apr. 23, 2003 (JP) 2003-118055

(51) Int. Cl. A01K 67/027; A61K 39/395

(52) U.S. Cl. 800/18; 424/145.1

(57) ABSTRACT

A method for evaluating an effect of an agent to be tested on alopecia, using a transgenic mouse to which an interleukin-18 gene under control of a keratinocyte promoter is introduced, or a method for evaluating an effect of an agent to be tested on skin, using the transgenic mouse, wherein the following (A) and/or (B) are used as an evaluation indicator. (A) spontaneous dermatitis (B) dermal inflammation at a time of a first administration of a phlogogenous material Using the transgenic mouse, the effect of an agent to be tested on dermatitis for which autoimmune dermatitis is a typical example and/or alopecia can be examined, and an agent for preventing/treating dermatitis or alopecia can be provided.

### TECHNICAL FIELD

The present invention relates to an evaluation method that can examine the effect of an agent to be tested on cutaneous symptoms for which autoimmune dermatitis is a typical example, or alopecia

### BACKGROUND ART

In recent years, the roles of interleukin-18 (hereinafter, referred to as “IL-18”) in autoimmune dermatitis including atopic dermatitis has attracted attention, and transgenic mice in which IL-18 genes under the control of a keratinocyte promoter are introduced have been produced. The transgenic mice are used to clarify the mechanism for inflammation occurrence.

In particular, a resent research employing transgenic mice in which IL-18 under the control of a keratinocyte promoter is expressed excessively in the skin has clarified that the IL-18 is deeply involved in the aggravation of contact dermatitis (which is regarded as one type of delayed type hypersensitivity [DTH]) induced by repetitive administration of a phlogogenous material such as trinitrochlorobenzene (TNCB), and has suggested that an antagonist of IL-18 can lead to creation of a new therapeutic agent for dermatitis.

Yusuke Kawase and eight others, “trinitrochlorobenzene-induced contact dermatitis can be enhanced in IL-18 transgenic mice”, p. 144, the program of the general meeting of the 26^{th} annual academic conference of Japanese Research Dermatology Society, September, 2001.

The inventors of the present invention attempted to employ these transgenic mice in screening of a new therapeutic agent for dermatitis, but this screening method requires repetitive administration (e.g., administration of one per week for six weeks), so that this method is difficult to perform in some aspects, for example, in that it takes time and bothering operation is required.

On the other hand, the relationship between the overexpression of IL-18 and alopecia is not known yet, and no one thought that a transgenic mouse to which an IL-18 gene was introduced could be used to evaluate alopecia or the like.

Furthermore, there is no specific report that a substance for preventing/treating dermatitis or alopecia has been screened using this transgenic mouse.

### DISCLOSURE OF INVENTION

The inventors of the present invention found that a transgenic mouse in which the IL-18 gene under the control of a keratinocyte promoter is introduced experienced the peak of spontaneous inflammation in a short time after birth, and that dermatitis occurs only by one administration of a phlogogenous material after the peak has subsided.

The inventors of the present invention found that a transgenic mouse in which the IL-18 gene under the control of a keratinocyte promoter is introduced starts to suffer from alopecia around the age of 15 weeks, and that the substance screened with this transgenic mouse can prevent/treat dermatitis or folliculitis, whichever may cause alopecia or the like.

The inventors of the present invention achieved the present invention based on the knowledge described above.

Therefore, it is an object of the present invention to provide an evaluation method that can examine the effect of an agent to be tested on cutaneous symptoms for which autoimmune dermatitis is a typical example in a simple manner and in a short time, to provide an evaluation method that can examine the effect of an agent to be tested on alopecia, provide a transgenic mouse that can be used for the evaluation about alopecia, and provide an agent for preventing/treating dermatitis and/or alopecia.

The objects of the present invention can be achieved by a method for evaluating an effect of an agent to be tested on skin, using a transgenic mouse in which an IL-18 gene under control of a keratinocyte promoter is introduced, characterized in that (A) spontaneous dermatitis and/or (B) dermal inflammation at the time of the first administration of a phlogogenous material are used as an evaluation indicator, the evaluation method characterized in that the spontaneous dermatitis reaches its peak on the 8^{th} to 12^{th} day after birth, and the evaluation method characterized in that the object of the evaluation is to screen an agent for preventing/treating autoimmune dermatitis.

The objects of the present invention can be achieved by an agent for preventing or treating dermatitis characterized by comprising a substance that suppresses a cell having an NK1.1 antigen.

The objects of the present invention can be achieved by an agent for preventing or treating alopecia characterized by comprising a substance that suppresses a cell having an NK1.1 antigen.

The objects of the present invention can be achieved by a transgenic mouse used to evaluate an effect of an agent to be tested on alopecia, in which an interleukin-18 gene under control of a keratinocyte promoter is introduced, or the transgenic mouse characterized in that the object of the evaluation is to screen an agent for preventing or treating alopecia.

Furthermore, the objects of the present invention can be achieved by a method for evaluating an effect of an agent to be tested on alopecia, using a transgenic mouse in which an interleukin-18 gene under control of a keratinocyte promoter is introduced, or the evaluation method characterized in that the object of the evaluation is to screen an agent for preventing/treating alopecia.

### BRIEF DESCRIPTION OF DRAWINGS

FIG. 1 is an example of a recombinant gene used in the present invention.

FIG. 2 is a graph showing the alopecia incidence of Tg (+) mice.

FIG. 3 is a photograph substituted for a drawing showing a pathological sample (state of a living thing) of the skin of a K5/IL-18 transgenic mouse that experienced the peak of spontaneous dermatitis on the 10^{th} day after birth. HE staining (×50)

FIG. 4 is a view showing occurrence of skin inflammation at the first administration of a phlogogenous material (B).

FIG. 5 is an image of dermal pathology of a group of Tg (+) mice to which [A] a control rabbit IgG antibody is administered (Reference Example 1). HE staining (×200)

FIG. 6 is an image of dermal pathology of a group of Tg (+) mice to which [B] an anti-asialo GM1 antibody is administered (Example 2). HE staining (×200)

FIG. 7 is an image of dermal pathology of a group of Tg (+) mice to which [A] a control antibody mouse IgG is administered (Reference Example 2). HE staining (×200)

FIG. 8 is an image of dermal pathology of a group of Tg (+) mice to which [C] an anti-mouse CD4 monoclonal antibody is administered (Comparative Example 1). HE staining (×200)

FIG. 9 is an image of dermal pathology of a group of Tg (+) mice to which [D] an anti-mouse CD8 monoclonal antibody is administered (Comparative Example 2). HE staining (×200)

FIG. 10 is an image of dermal pathology of a group of Tg (+) mice to which [B] an anti-mouse NK1.1 monoclonal antibody is administered (Example 3). HE staining (×200)

FIG. 11 is an image of dermal pathology of a group of Tg (+) mice to which [A] a control vehicle mouse is administered (Reference Example 3). HE staining (×200)

FIG. 12 is an image of dermal pathology of a group of Tg (+) mice to which [B] Dexamethasone is administered (Comparative Example 3). HE staining (×200)

FIG. 13 is an image of dermal pathology of a group of Tg (+) mice to which [A] a control vehicle mouse is administered (Reference Example 4). HE staining (×200)

FIG. 14 is an image of dermal pathology of a group of Tg (+) mice to which [B] FK506 is administered (Example 4). HE staining (×200)

FIG. 15 is an image of dermal pathology of a group of Tg (+) mice to which [B] an anti-Ly49D monoclonal antibody is administered (Example 5). HE staining (×200)

FIG. 16 is an image of dermal pathology of the face of a female Tg (+) mouse at the age of 9 months. HE staining (×200)

FIG. 17 is an image of dermal pathology of the back of a female Tg (+) mouse at the age of 9 months. HE staining (×200)

### DEFINITIONS OF EXPRESSIONS

hK5 promoter: human keratinocyte 5 promoter
SP: signal peptide
mature mIL-18: mature mouse IL-18 gene
pA: poly A sequence
K5TG: K5/IL-18 transgenic B6 mouse
WT: wild-type B6 mouse
KS/IL-18Tg: K5/IL-18 transgenic B6 mouse
Ig/IL-18Tg: Ig/IL-18 transgenic B6 mouse
TG: KS/IL-18Tg, or Ig/IL-18Tg
vehicle: acetone/olive oil (4:1, v/v)
croton oil: croton oil solution with 2% (v/v) acetone/olive oil (4:1, v/v)

### BEST MODE FOR CARRYING OUT THE INVENTION

### Transgenic Mouse

A transgenic mouse used in the evaluation method of the present invention can be produced, for example, in the following manner.

Regarding the mice, for example, C57BL/6N mice (B6 mice), Balb/c mice and the like are used preferably, and among these, B6 mice are preferable.

A recombinant gene that can be introduced to the mouse includes a keratinocyte promoter and an IL-18 gene that is under the control thereof.

As the keratinocyte promoter, a human keratinocyte 5 promoter (hereinafter, referred to as “hK 5 promoter”) and the like can be used.

In addition, it is preferable to introduce a signal peptide gene for accelerating release of a transducing gene to the outside of a cell or a poly A sequence that is useful for detecting an expressed gene or the like to the recombinant gene.

As the signal peptide, for example, immunoglobulin (hereinafter, referred to as “Ig”) κ-chain signal peptide of a mouse or the like can be used.

As the poly A sequence, a bovine-derived poly A sequence or the like can be used.

As a method for producing the recombinant gene including the above, a known method for gene recombination can be used. For example, the following manner can be used.

A mature IL-18 cDNA having a signal peptide is obtained by PCR, using a signal peptide taken out from the V-J2-C region of mouse Igκ-chain and pro-IL-18 cDNA of a mouse.

Next, subdloning is performed with a pCR2.1 vector, and then subdloning is performed with a pcdEF3 vector including a promoter of human elongation factor 1α and a bovine-derived poly A sequence to produce pEF-IL-18SP.

A DNA fragment of the pEF-IL-18SP that is cleaved at a restriction enzyme cleavage site of KpnI/BbsI is subeloned at the NotI site of a pBSK vector including a human K5 promoter.

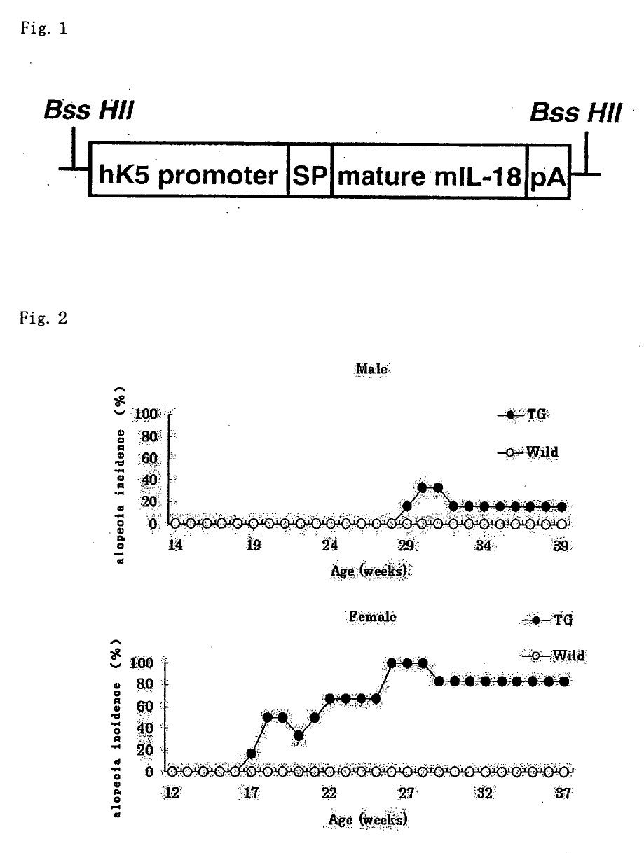

A linear DNA fragment (K5/SP/IL-18/polyA) can be obtained by cleaving the vector at the BssHII restriction enzyme cleavage site (FIG. 1).

A known gene introduction method can be used as the method for introducing the recombinant gene to a mouse. For example, the recombinant gene can be introduced to a mouse by crossbreeding a mouse whose fertilized egg has been injected with a recombinant gene obtained in the above described manner with a wild-type mouse, and selecting a mouse expressing IL-18 of its progeny. Examples of the selection method include PCR analysis using genome DNA of the tail, ELISA analysis of mature IL-18 in blood serum, and Western blotting analysis of mature IL-18 of the skin.

In the thus obtained transgenic mice, folliculitis or chronic dermatitis images were observed after 20 weeks, and it was confirmed by the inventor of the present invention for the first time that in almost all female mice and 20 to 40% of male mice, topical alopecia occurred (FIG. 2).

Therefore, it is possible to evaluate the effect of an agent to be tested on dermatitis that causes alopecia or the like and to screen hair tonics, hair restorers or the like, using the transgenic mice to which interleukin-18 gene under the control of a keratinocyte promoter is introduced.

Thus, it is the first time that the transgenic mice in which interleukin-18 gene under the control of a keratinocyte promoter is introduced are used in a method for evaluating the effect of an agent to be tested on alopecia, and the transgenic mice of the present invention are novel as transgenic mice that used to evaluate the effect of an agent to be tested on alopecia.

### Method for Evaluating the Effect of an Agent to be Tested on Dermatitis

The phenomena used as evaluation indicators in the method for evaluating the effect of an agent to be tested on the skin of the present invention are the following two inflammatory reactions.
(A) spontaneous dermatitis
(B) dermal inflammation at the time of the first administration of phlogogenous material

The evaluation of the present invention is performed by confirming the effect of an agent to be tested in one or both of the above two items (A) and (B).

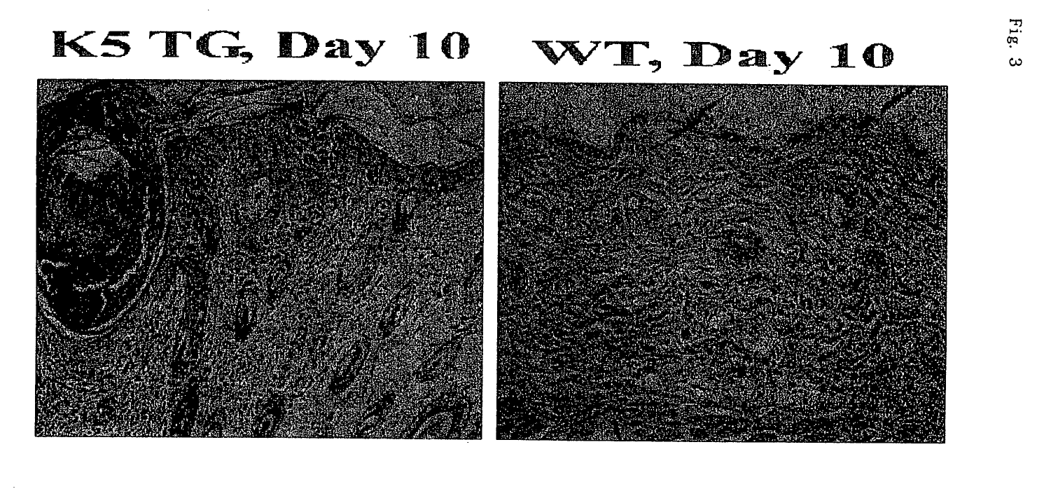

The spontaneous dermatitis (A) refers to an inflammation that reaches its peak approximately on the 8^{th} to 12^{th} day after birth of an IL-18-introduced mouse and disappears after about four weeks and that has been identified by the inventors of the present invention for the first time. Pathologically speaking, in this inflammation, acanthosis (epidermal thickening), hyperkeratosis, and inflammatory cell infiltrations that neutrophilic leukocytes are contained in dermis can be observed in dermal tissue, a slight sponge state is present in the epidermal base, and abscess formation in the cuticle due to epidermal neutrophilic infiltrations is observed (FIG. 3).

Therefore, the effect of preventing/treating the inflammation of an agent to be tested can be confirmed in a significantly shorter time than conventional cases by using the confirmation of these symptoms during about 8 to 12 days after birth as the indicator, so that it is possible to screen agents for treating or preventing the inflammation.

The dermal inflammation at the time of the first administration of phlogogenous material (B) refers to a dermal inflammation induced by administrating a phlogogenous material for the first time, for example, after the peak of spontaneous dermatitis, and while the peak of the spontaneous dermatitis still remains or after that symptom has disappeared for the moment.

It was confirmed that the overexpression of IL-18 is involved in the occurrence of both of these symptoms (A) and (B), and that these symptoms have the same mechanism by which the inflammation occurs as that of contact hypersensitivity caused by repetitive administration of a phlogogenous material over 6 weeks that has been reported before.

Therefore, the evaluation methods of the present invention using (A) and (B) as the indicators also can be utilized as a rapid and simple method for evaluating contact hypersensitivity. In other words, by using (A) as an indicator, screening of agents for preventing/treating contact hypersensitivity, which conventionally required repetitive administration for as long a time as a further 6 weeks after dermatitis has subsided for the moment, can be performed in a shorter time such as about 8 to 12 days after birth. Moreover, by using (B) as an indicator, an agent to be tested can be evaluated in a simple manner (by using as an indicator dermatitis caused by treating with a phlogogenous material such as croton oil after dermatitis has disappeared temporarily). Furthermore, screening can be performed by using both of (A) and (B) as indicators.

The evaluation method of the present invention can be performed as a method for screening agents for preventing/treating autoimmune dermatitis. The screening can be performed, for example, by the procedures of administrating an agent to be tested in either stage, before or after the occurrence of the peak of spontaneous dermatitis, and comparing mice without administration (control) in terms of the presence or the absence of peak occurrence, the size of the peak, the speed of peak disappearance or the like.

### Method for Evaluating the Effect of an Agent to be Tested on Alopecia

The method for evaluating the effect of an agent to be tested on alopecia can be performed as a method for screening agents for preventing/treating alopecia. The screening can be performed, for example, by the procedures of administrating an agent to be tested in either stage, before or after occurrence of alopecia, and comparing mice without administration (control) in terms of the presence or the absence of peak occurrence, the size of the peak, the speed of peak disappearance or the like.

### Agents for Preventing/Treating Dermatitis and/or Alopecia of the Present Invention

An active component of an agent for preventing/treating dermatitis or an agent for preventing/treating alopecia of the present invention is a substance that suppresses cells having the NK1.1 antigen.

Examples of the cells having an NK1.1 antigen include NK cells and NKT (NK-T) cells.

Examples of the substance that suppresses the cells having an NK1.1 antigen used in the present invention include antibodies against cells having an NK1.1 antigen such as anti-NK cell antibodies and anti-NKT cell antibodies, immunosupressants such as soluble HLA-class I proteins and FK 506, and inhibitors of molecules that participate in signal transduction of NK cells such as DAP12 and SHP present downstream of NK cell receptors. The antibodies against the cells having an NK1.1 antigen are preferable.

As the antibodies against the cells having an NK1.1 antigen, in general, (1) antibodies that suppress cells having an NK1.1 antigen such as antibodies identifying an asialo GM 1 antigen, and anti-killer inhibitory receptor (KIR) antibodies including antibodies identifying an NK1.1 antigen, (2) antibodies that activate NK cells, and (3) antibodies against antigens that activate NK cells such as Ly49D (anti-Ly49D antibodies) can be used. In the present invention, the antibodies that suppress NK cells (1) are preferable.

As the antibodies identifying an asialo GM 1 antigen used in the present invention, either monoclonal or polyclonal, or chimeric antibodies or humanized antibodies can be used.

More specifically, for example, there is an anti-asialo GM1 antibody, which is a polyclonal antibody, that can be obtained from the blood serum of a rabbit that has been immunized with asialo GM1 antigen, which is a glycolipid, as an antigen molecule. However, in order to prevent or treat dermatitis or alopecia of human beings, chimeric antibodies or humanized antibodies obtained by using antibodies that are immunized with an asialo GM1 antigen derived from human beings are preferable.

As the antibodies identifying an NK1.1 antigen used in the present invention, either monoclonal or polyclonal, or chimeric antibodies or humanized antibodies can be used.

More specifically, for example, there is an anti-NK1.1 antibody (BD Bioscience Pharmingen Technical Data Sheet Catalog Number 553161, revision No. 011, published on October 2002), which is a monoclonal antibody, that can be obtained from a mouse that has been immunized with a mouse NK cell as an antigen molecule, and an isoantibody identifying a mouse NK1.1 antigen (NKR-1B, NKR-P1C). However, in order to prevent or treat dermatitis or alopecia of human beings, chimeric antibodies or humanized antibodies obtained by using antibodies identifying human NK1.1 antigen (CD161) obtained by immunizing human NK cells are preferable.

The agent for preventing/treating dermatitis or agent for preventing/treating alopecia of the present invention can be administered systemically by injection or the like or administered topically. However, topical administration in which an agent is administered directly and topically to the skin where dermatitis can occur easily or the skin of scalp or the like is preferable. For systemic administration, intravenous administration or the like can be performed. For topical administration, subcutaneous or intramuscular administration or application of an ointment, a patch or the like onto the skin or the skin of scalp can be performed.

An injection agent can be prepared by an ordinary method. That is, an injection agent can be prepared by dissolving or suspending an anti-asialo GM1 antibody, an anti-NK1.1 antibody in a buffer solution such as a physiological saline solution or PBS and lyophilizing the solution aseptically.

Furthermore, an ointment or a patch can be prepared by an ordinary method.

Examples of dosage forms include cream, salve, lotion, emulsion, solution, gel, powder, and solid such as a stick, and examples of commercial product forms include various skin cosmetics such as packs, lotions (cosmetic lotions), and hair cosmetics for hair such as shampoos, rinses, hair treatments, hair tonics, and hair-restorers.

Various auxiliaries that can be commonly added to medicaments, such as stabilizers, antiseptic agents or soothing agents can be added appropriately to the agent for preventing/treating dermatitis or the agent for preventing/treating alopecia of the present invention, if necessary.

The agent for preventing/treating dermatitis or the agent for preventing/treating alopecia of the present invention can be used together with other components as raw materials of cosmetics such as skin cosmetics, unregulated drugs, designated unregulated drugs, drugs for external use, or the like.

As the components that are used together, any materials can be used, as long as they are commonly used as raw materials of cosmetics, unregulated drugs, designated unregulated drugs, drugs for external use, or the like.

More specifically, for example, components such as surfactants such as anionic surfactants, ampholytic surfactants or nonionic surfactants, mucilaginous agents, oil, powdery substances (pigment, dye, resin), antiseptic agents, aroma chemicals, moisturizing agents, bioactive components, salts, solvents, antioxidants, chelating agents, pearlizing agents, neutralizing agents, pH regulators, insect repellents, enzyme or the like can be used.

These components can be contained as appropriate in cosmetics, unregulated drugs, designated unregulated drugs, drugs for external use or the like in the range that does not inhibit the effect of the agent for preventing/treating dermatitis or the agent for preventing/treating alopecia of the present invention.

Furthermore, the agent for preventing/treating dermatitis or the agent for preventing/treating alopecia of the present invention can be used as a pharmaceutical in which a drug delivery system (DDS) is used.

The dosage amount depends on the disease, the condition, the route of administration, the age and the weight of a patient, the type of active components, and the administration form, but in general, an amount of a substance that suppresses cells having an NK1.1 antigen, which is an active component, of 0.001 to 10 mg/kg per day for an adult is administered once or in 2-3 divided doses. The agent is not necessarily administered every day, but can be administered every few days, for example, once every one day to four days.

### EXAMPLES

The present invention will be described more specifically by way of examples. Prior to the examples of the evaluation method, a lineage causing (B) dermal inflammation at the time of the first administration of a phlogogenous material (control) was established. This is one of the evaluation indicators used in the method for evaluating the effect of an agent to be tested on the skin of the present invention.

(B) Occurrence of Dermal Inflammation at the Time of the First Administration of a Phlogogenous Material

Ten μl of croton oil solution in which 2% (v/v) of croton oil was dissolved in acetone/olive oil (4:1, v/v) was applied to the right ear of each of male K5/IL-18 transgenic B6 mice (at the age of 11 weeks), Ig/IL-18 transgenic B6 mice (at the age of 10-12 weeks) (T. Hoshino, J, Immunology, 2001, 7014-7018) and B6 mice (wild-type) at the same age, and 10 μl of acetone/olive oil (4:1, v/v) was applied to the left ear as control. The thickness of the ears was measured after 0, 6, 24 and 48 hours, and 7 days.

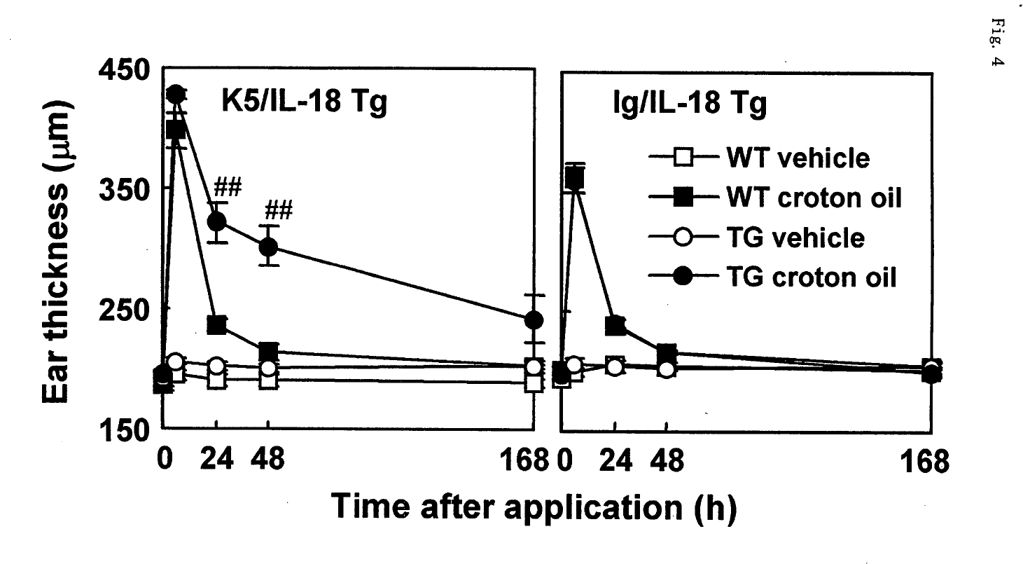

FIG. 4 shows the results.

As shown in FIG. 4, in the K5/IL-18 transgenic B6 mice, significant swelling of the auricle was observed until after 48 hours. On the other hand, in the Ig/IL-18 transgenic B6 mice and the wild-type mice, after 48 hours, the swelling of the auricle had disappeared.

### Example 1

### Screening of Agents for Preventing/Treating Autoimmune Dermatitis

A treatment was performed in the same manner as in (B) described above, except that a solution in which an agent to be tested (candidate compound of a preventive or therapeutic agent) was dissolved or suspended in an appropriate solvent (the same solvent as that applied to the left ear) such as acetone/olive oil (4:1, v/v) was applied to the right ear as appropriate before, at the same time or after the application of 2% (v/v) croton oil solution. Then, swelling of the auricle was measured after 24 hours, and the suppression effect of the swelling of auricle by the agent to be tested was determined by comparing it with the control lineage.

Next, the agent of preventing/treating dermatitis of the present invention will be described more specifically by way of examples. Five to seven mice were grouped into one group, and agents to be tested of examples, comparative examples, reference examples or the like were administered to each group.

### Example 2

### Dermatitis Suppression by Administrating an Anti-Asialo GM1 Antibody

[A] control rabbit IgG antibody (Sigma) in an amount of 2 mg or [B] anti-asialo GM1 antibody (Wako) in an amount of 2 mg that was dissolved in 0.1 mL of PBS was administered intraperitoneally to K5/IL-18 Tg (IL-18 transgene (+), hereinafter, referred to as “Tg (+)”) mice within one day after birth.

Ten days after the administration of the antibodies, the skins of the backs of the mice of the two groups A and B was obtained, and fixed in 20% buffered formalin. The dermatitis was histopathologically examined by HE staining.

The observation of the appearance 10 days after the administration of the antibody indicated that in [A] administration group (Reference Example 1), dermatitis occurred, whereas in [B] administration group (Example 2), dermatitis was suppressed.

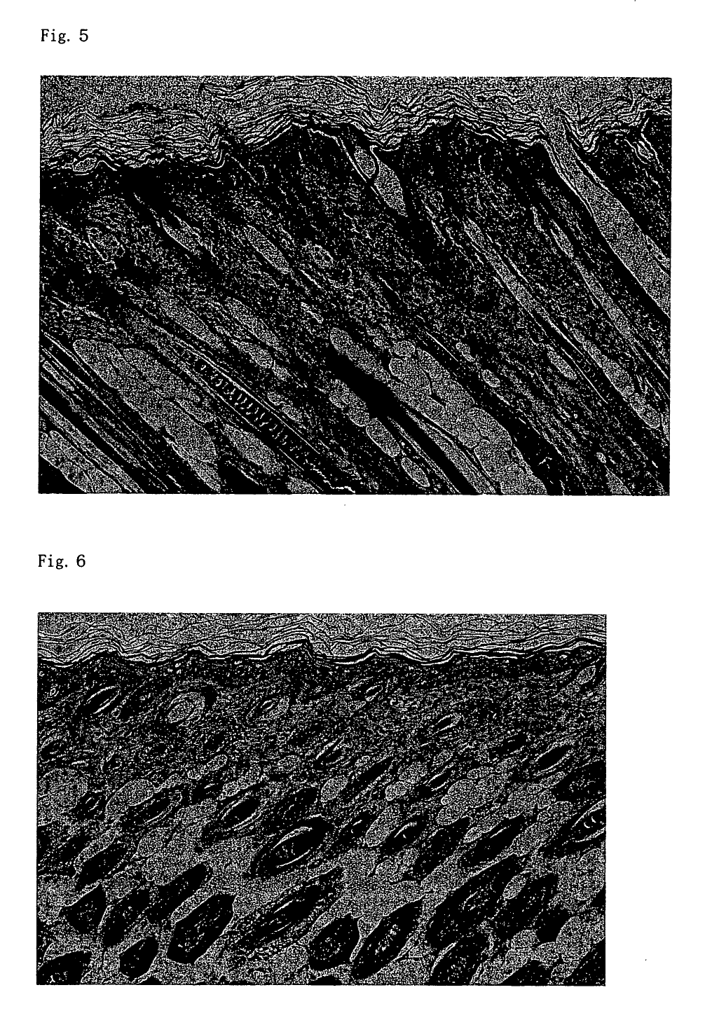

When their pathological images were observed, in [A] administration group (Reference Example 1), cuticle thickening, epidermal thickening, and neutrophilic leukocyte-dominant infiltrations to dermis were seen in the Tg (+) mice, and in the control antibody rabbit IgG, it was confirmed that the dermatitis of the Tg (+) mice was not suppressed (FIG. 5).

On the other hand, in the [B] administration group (Example 2), cuticle thickening, epidermal thickening, lymphocyte infiltration to dermis and the like were suppressed, and it was confirmed that the dermatitis of the Tg (+) mice was suppressed by the anti-asialo GM1 antibody (FIG. 6).

As a result, the anti-asialo GM1 antibody was found to be effective as an agent for preventing/treating dermatitis, and also effective as an agent for preventing/treating alopecia that is caused by dermatitis.

### Example 3, Comparative Examples 1 and 2

### Dermatitis Suppression by Administrating Anti-NK1.1 Antibody

[A] control mouse IgG antibody (Sigma) in an amount of 0.5 mg, [B] anti-mouse NK1.1 monoclonal antibody (PK136) in an amount of 0.5 mg, [C] anti-mouse CD4 monoclonal antibody (GK1.5) in an amount of 0.5 mg, or [D] anti-mouse CD8 monoclonal antibody (2.43) in an amount of 0.5 mg that was dissolved in 50 μL of PBS was administered intraperitoneally to Tg (+) mice within one day after birth.

Ten days after the administration of the antibodies, the skins of the backs of the mice of the four groups A to D was obtained, and fixed in 20% buffered formalin. The dermatitis was histopathologically examined by HE staining.

The observation of the appearance 10 days after the administration of the antibodies indicated that in [A] administration group (Reference Example 2), [C] administration group (Comparative Example 1), and [D] administration group (Comparative Example 2), dermatitis occurred, whereas in [B] administration group (Example 3), dermatitis was suppressed.

Histopathological analysis revealed that in the [A] administration group (Reference Example 2), cuticle thickening, epidermal thickening, neutrophilic leukocyte-dominant infiltration to dermis and slight abscess in the corneum were seen in the Tg (+) mice. It was confirmed that control mouse IgG antibody did not suppress the dermatitis (FIG. 7).

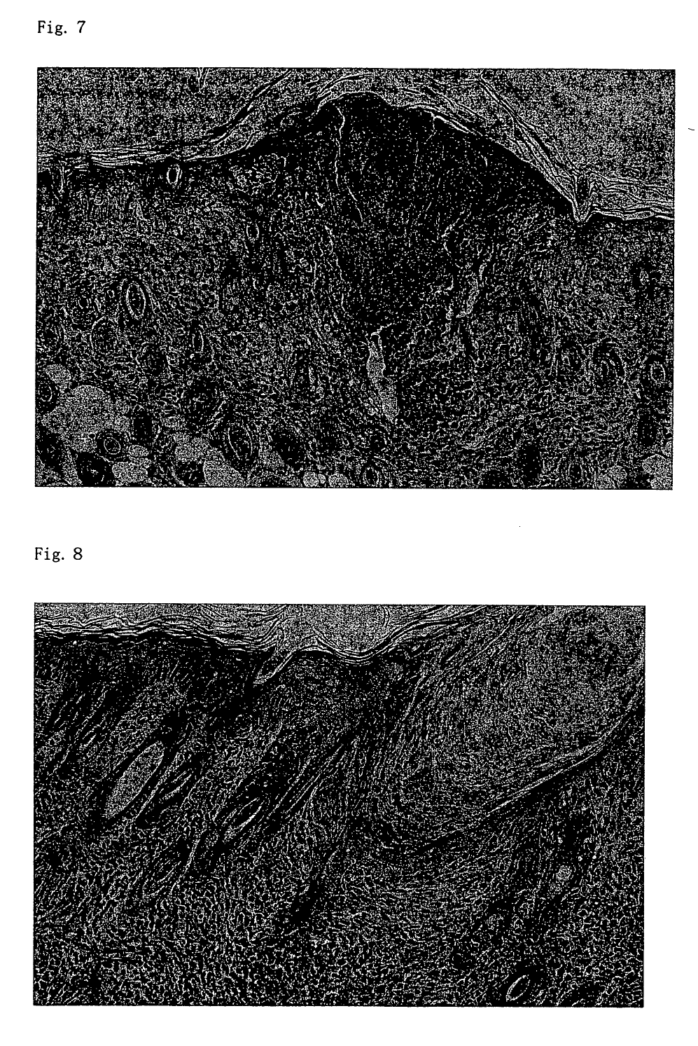

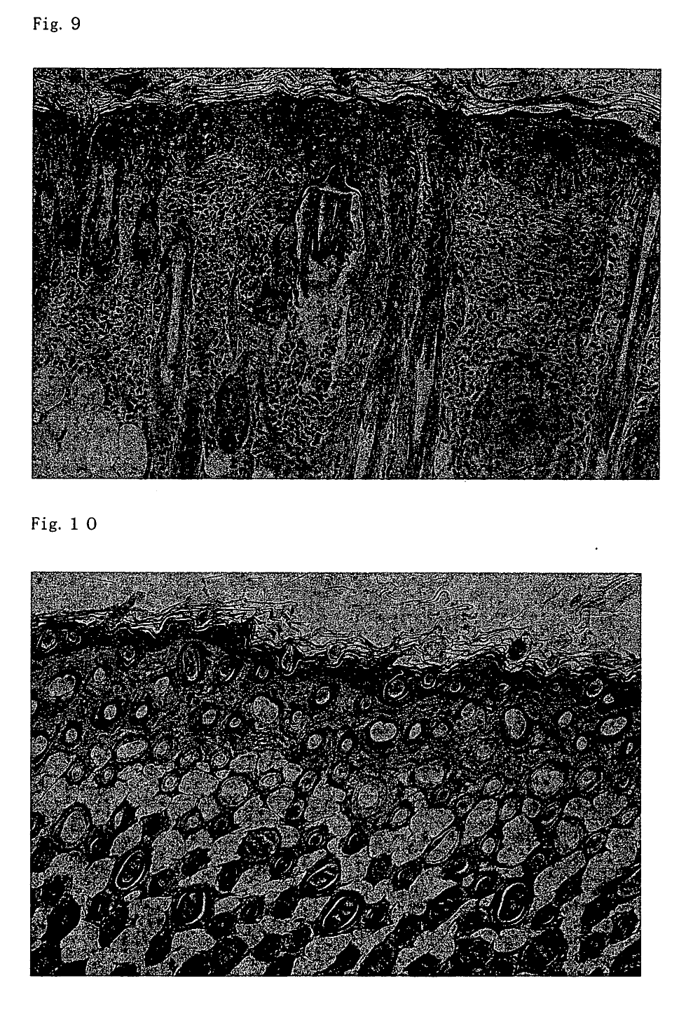

In the [C] administration group (Comparative Example 1), cuticle thickening, epidermal thickening, neutrophilic leukocyte-dominant infiltration to dermis and slight abscess in the corneum were seen in the Tg (+) mice, and this group had tendency to be worse than the [A] administration group, and it was confirmed that the anti-CD4 antibody did not suppress the dermatitis of the mice (FIG. 8). Furthermore, also in the [D] administration group (Comparative Example 2), cuticle thickening, epidermal thickening, neutrophilic leukocyte-dominant infiltration to dermis and slight abscess in the corneum were seen in the Tg (+) mice (FIG. 9).

On the other hand, in the [B] administration group (Example 3), cuticle thickening, epidermal thickening, lymphocyte infiltration to dermis and the like were suppressed, and it was confirmed that the dermatitis of the Tg (+) mice was suppressed significantly by the anti-NK1.1 antibody (Example 3: FIG. 10).

As a result, the anti-NK1.1 antibody was found to be effective as an agent for preventing/treating dermatitis, and also effective as an agent for preventing/treating alopecia that is caused by dermatitis.

### Comparative Example 3

### Dermatitis Suppression by Administrating Steroid [Dexamethasone]

[A] 0.5% CMC-Na (Kanto Chemical, Catalogue No. 37140-02)/PBS in an amount of 100 μL (vehicle) that was alone or [B] 1% dexamethasone (10 mg/mL) that was dissolved in 100 μL of 0.5% CMC-Na/PBS was applied to the skin of the backs of Tg (+) mice within three days after birth for consecutive seven days.

Seven days after the first administration (that is, one day after the last administration and 10 days after birth), the skins of the backs of the mice of the two groups was obtained, and fixed in 20% buffered formalin. The dermatitis was histopathologically examined by HE staining.

The observation of the appearance 8 days after application of dexamethasone indicated that in [A] administration group (Reference Example 3), dermatitis occurred, whereas in [B] administration group (Comparative Example 3), dermatitis was suppressed.

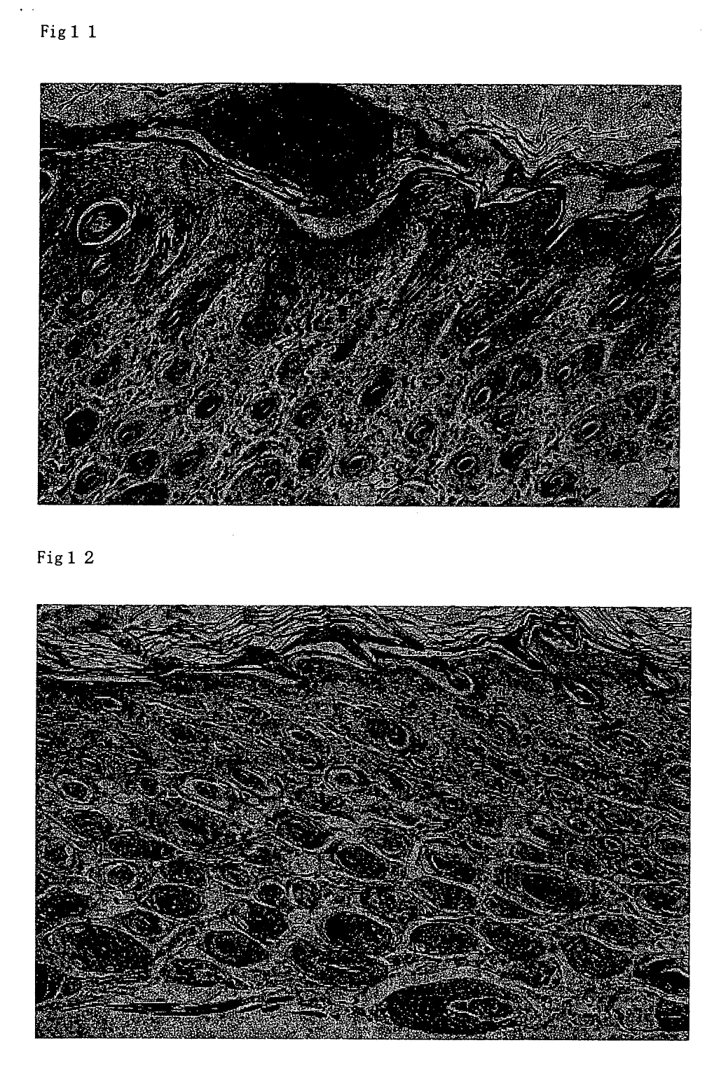

When their pathological images were observed, in the [A] administration group (Reference Example 3), cuticle thickening, epidermal thickening, neutrophilic leukocyte-dominant infiltration to dermis and slight abscess in the corneum were seen in the Tg (+) mice, and it was confirmed that the vehicle did not suppress the dermatitis of the Tg (+) (FIG. 11).

On the other hand, in the [B] administration group (Comparative Example 3), cuticle thickening, epidermal thickening, and lymphocyte infiltration to dermis were suppressed, and it was confirmed that the dermatitis was suppressed to some extent (FIG. 12). However, the suppression effect was not so large as that of the anti-NK1.1 antibody of Example 2, and especially the cuticle thickening was almost not suppressed. Furthermore, about a half of the dexamethasone-treated group [B] died from side effects.

The results revealed that the steroid agent suppresses dermatitis to some extent, but had a big problem in practice because the side effects were strong.

### Example 4, Reference Example 4

### Dermatitis Suppression by Administrating FK506

[A] 0.5% CMC-Na (Kanto Chemical, Catalogue No. 37140-02)/PBS in an amount of 100 μL (vehicle) that was alone or [B] 1% FK506 (10 mg/mL, supplied by Fujisawa Pharmaceutical) that was dissolved in 100 μL of 0.5% CMC-Na/PBS was applied to the skin of the backs of Tg (+) mice within three days after birth for consecutive seven days. Seven days after the first administration (that is, one day after the last administration and 10 days after birth), the skin of the backs of the mice of the two groups was removed, and fixed in 20% buffered formalin. The dermatitis was histopathologically examined by HE staining.

The observation of the appearance 7 days after the first administration (that is, one day after the last administration and 10 days after birth) indicated that in [A] administration group (Reference Example 4), dermatitis occurred, whereas in [B] administration group (Example 4), dermatitis was suppressed.

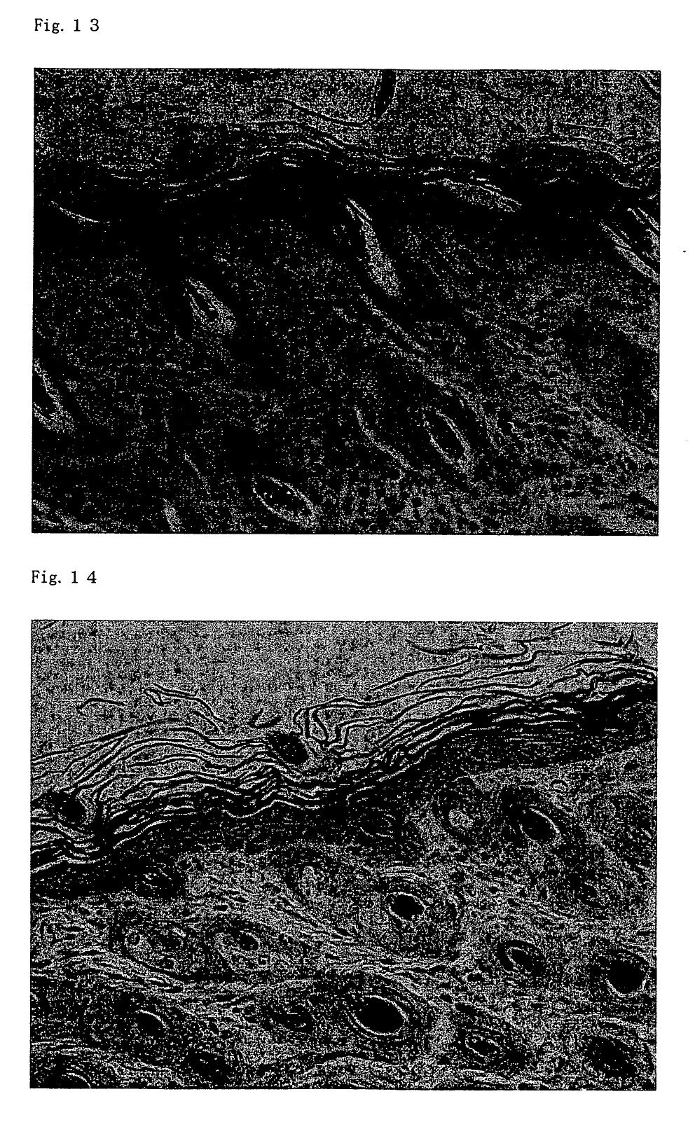

Pathologically speaking as well, in the [B] administration group (Example 4), infiltration of inflammatory cells to dermis was also suppressed more than in the [A] administration group (Reference Example 4) (Reference Example 4: FIG. 13/Example 4: FIG. 14).

In other words, the FK506 application was found to suppress dermatitis, though not so much as the anti-asialo GM1 antibody (Example 2) or the anti-NK1.1 antibody (Example 3).

### Example 5

### Dermatitis Suppression by Administrating Anti-Ly49D Antibody

Tg (+) mice within one day after birth were used. An anti-Ly49D antibody (4E5) in an amount of 0.5 mg that was dissolved in 50 μL of PBS was administered intraperitoneally to the Tg (+) mice.

Ten days after the administration of the antibody, the skin of the backs of the mice was removed, and fixed in 20% buffered formalin. The dermatitis was histopathologically examined by HE staining.

The observation of the appearance 10 days after the administration of the antibody indicated that in the administration group of the anti-Ly49D antibody, dermatitis was suppressed.

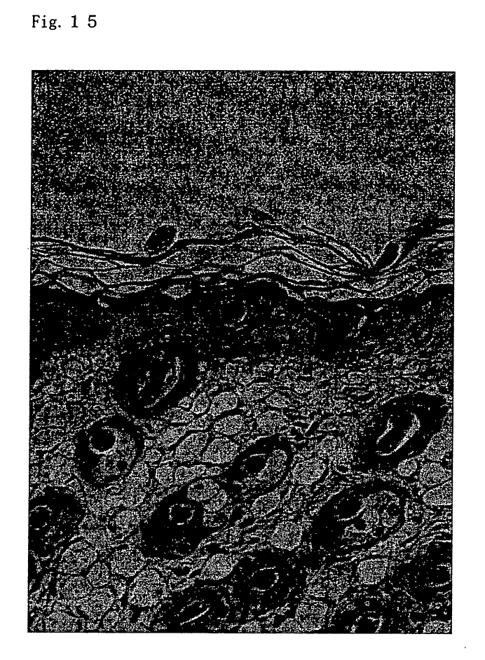

When their pathological images were observed, in the administration group of the anti-Ly49D antibody, cuticle thickening, epidermal thickening, neutrophilic leukocyte-dominant infiltration to dermis and slight abscess in the corneum were only slightly seen in the Tg (+) mice (FIG. 15).

These results revealed that the agent for preventing/treating dermatitis or alopecia of the present invention has better suppression effects on dermatitis which may cause alopecia, but has no side effects and are very useful as an excellent preventive/therapeutic agent.

### Test Example

The following test example confirmed that dermatitis was concerned with alopecia. As shown in FIG. 2 described above, in the Tg (+) mice, folliculitis or chronic dermatitis images were observed after 20 weeks, and it was confirmed that in almost all the female mice and 20 to 40% of the male mice, topical alopecia occurred.

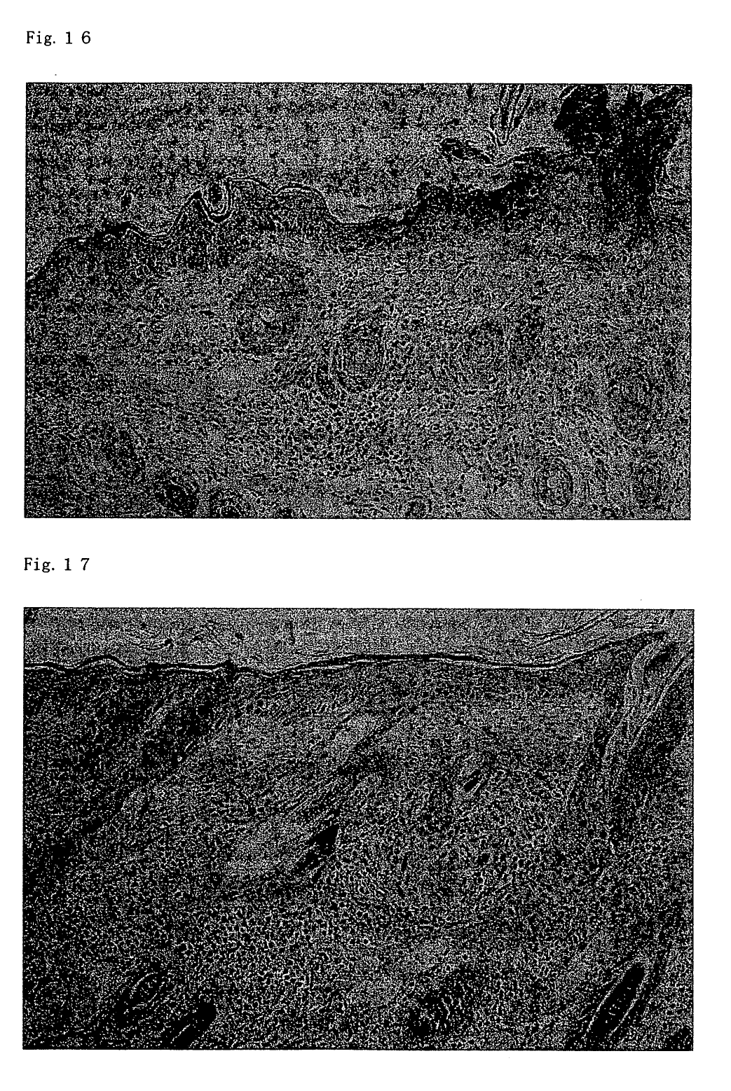

Furthermore, in order to examine the pathological images of the skin of the female Tg (+) mice at the age of 9 months, the skin of the face and the back of the mice was removed and fixed in 20% buffered formalin, and the dermatitis was histopathologically examined by HE staining. Then, it was confirmed that trichitis occurred (face: FIG. 16, back: FIG. 17).

The trichitis of FIGS. 16 and 17 was profound folliculitis in which strong inflammatory cell infiltration was present in the periphery of the hair follicle (perifolliculitis), and in addition, inflammatory cell infiltration was partially present also in the squamous epithelium forming the hair follicle. In FIG. 16, a cuticle portion of the skin was lost and ulcerated due to serious dermatitis. In FIG. 17, alopecia and dermatitis with significant inflammatory cell infiltration and folliculitis in the hair root portion occurred.

The above-described results indicated that the transgenic mouse of the present invention can be used to evaluate the effect of an agent to be tested on alopecia. The above-described results indicated that dermatitis is suppressed by the agent for preventing/treating dermatitis of the present invention, so that trichitis also can be prevented and alopecia can be prevented.

### INDUSTRIAL APPLICABILITY

The evaluation method of the present invention makes it possible to evaluate an agent to be tested on contact dermatitis in such a short time as 8 to 12 days after birth, as opposed to conventionally requiring as long a time as a further 6 weeks for repetitive administration of a phlogogenous material, without administrating a phlogogenous material. Or the bothering operation of repetitive administration of a phlogogenous material is eliminated so that evaluation can be performed in a simple manner.

Furthermore, the agent for preventing/treating dermatitis of the present invention can prevent/treat effectively the whole range of dermal disorders including dermatitis such as alopecia, psoriasis, autoimmune diseases, and allergic dermatitis, especially, dermal disorders derived from overexpression of IL-18 among these.

Furthermore, the transgenic mouse of the present invention makes it possible to screen agents for preventing/treating alopecia. Furthermore, the evaluation method of the present invention makes it possible to examine the effect of an agent to be tested on alopecia.

Furthermore, the agent for preventing/treating dermatitis or alopecia of the present invention is effective for preventing/treating dermatitis or alopecia and has the advantage that the safety is higher than that of hormone agents such as steroid.

1-11. (canceled)

12. A method of preventing or treating dermatitis and/or alopecia, comprising administering to a patient an effective amount of a composition comprising at least one antibody against NK cells.

13. The method of claim 12, wherein the antibody is an antibody that suppresses NK cells.

14. The method of claim 13, wherein the antibody is an antibody identifying an NK1.1 antigen.

15. The method of claim 14, wherein the antibody is an antibody identifying human NK1.1 antigen.

16. The method of claim 13, wherein the antibody is an antibody identifying an asialo GM1 antigen.

17. The method of claim 13, wherein the at least one antibody is selected from the group consisting of antibodies identifying an NK1.1 antigen and antibodies identifying an asialo GM1 antigen.

18. The method of claim 12, wherein the antibody is an antibody against an antigen that activates NK cells.

19. The method of claim 18, wherein the antibody is anti-Ly49D antibody.

20. The method of claim 12, wherein the antibody is selected from the group consisting of antibodies identifying an NK1.1 antigen, antibodies identifying an asialo GM1 antigen, and anti-Ly49D antibodies.

21. The method of claim 12, wherein the composition is administered topically.

22. The method of claim 21, wherein the composition is administered directly onto the skin.

23. The method of claim 21, wherein the composition is administered subcutaneously.

24. The method of claim 21, wherein the composition is administered intramuscularly.

25. The method of claim 12, wherein the composition is administered systemically.

26. The method of claim 25, wherein the composition is administered by injection.

27. The method of claim 25, wherein the composition is administered intravenously.

28. The method of claim 12, which is a method of preventing or treating dermatitis.

29. The method of claim 12, which is a method of preventing or treating alopecia.

30. The method of claim 29, wherein the alopecia is caused by dermatitis.

31. The method of claim 12, which is a method of preventing or treating psoriasis.

32. The method of claim 12, which is a method of preventing or treating autoimmune dermatitis.

33. The method of claim 12, which is a method of preventing or treating allergic dermatitis.

34. The method of claim 12, which is a method of preventing or treating atopic dermatitis.

35. The method of claim 12, which is a method of preventing or treating dermal disorders derived from overexpression of IL-18.
